(19) United States
(12) Patent Application Publication
(10) Pub. No.: US 2013/0273535 A1
(43) Pub. Date: Oct. 17, 2013

(54) NDM-1 POLYMERASE CHAIN REACTION (PCR) ASSAY

(71) Applicant: Carmelo Volpe, Herfordshire (GB)

(72) Inventor: Carmelo Volpe, Herfordshire (GB)

(73) Assignee: SMITHS DETECTION-WATFORD LTD., Watford (GB)

(21) Appl. No.: 13/817,024

(22) PCT Filed: Aug. 16, 2011
(86) PCT No.: PCT/IB11/02667

§ 371 Date: Jun. 28, 2013

Related U.S. Application Data
(60) Provisional application No. 61/374,385, filed on Aug. 17, 2010.

(51) Int. Cl. C12Q 1/68

(52) U.S. Cl. CPC C12Q 1/689
USPC 435/6.11

(57) ABSTRACT

Provided herein are compositions, methods, and kits for detection, identification, and analysis of NDM-1 variant nucleic acid. In particular, provided herein are kits, compositions, and methods for the detection, identification, and analysis of the NDM-1 variant nucleic acid and bacteria o other organisms carrying the NDM-1 variant nucleic acid.

### CROSS-REFERENCE TO RELATED APPLICATIONS

This application claims priority to U.S. Provisional Patent Application Ser. No. 61/374,385, filed Aug. 17, 2010, which is herein incorporated by reference in its entirety.

### FIELD

Provided herein are compositions, methods, and kits for detection, identification, and analysis of NDM-1 variant nucleic acid. In particular, provided herein are kits, compositions, and methods for the detection, identification, and analysis of the NDM-1 variant nucleic acid and bacteria or other organisms carrying the NDM-1 variant nucleic acid.

### BACKGROUND

Antibiotic resistant bacteria harbor genes which provide them with the ability to survive exposure to one or more antibiotics. Antibiotic resistance genes can often be laterally transferred between bacteria of the same or different species by conjugation, transduction, or transformation. Thus a gene for antibiotic resistance which had evolved via natural selection may be shared. Many antibiotic resistance genes reside on plasmids, facilitating their transfer. Evolutionary stress, such as exposure to antibiotics, then selects for the antibiotic resistant trait. If a bacterium carries several resistance genes or a gene which provides resistance to multiple classes of antibiotics, it is called multiresistant or, informally, a superbug.

The metallo-β-lactamases (MBLs) have emerged as one of the most worrisome resistance mechanism due to their capacity to hydrolyze all β-lactam agents, excluding aztreonam, including the carbapenems; and because their genes are carried on highly mobile elements allowing easy dissemination of the genes. The emergence of these enzymes compromises the effectiveness of treatments of bacterial infections, and is a harbinger for worsening antibiotic resistance to come (Zavascki et al. Critical Care 2006. 10:R114., herein incorporated by reference in its entirety).

### SUMMARY

Provided herein are compositions, kits, and methods for detection and analysis of multiresistant organisms. In particular, compositions, kits, and methods are provided for the detection and analysis of pathogenic organisms that express or harbor sequences that encode enzymes that provide antibiotic resistance, including those that express or harbor nucleic acid encoding variant NMD-1 sequences.

In some embodiments, herein are provided methods for detecting, identifying, or analyzing NDM-1 variants of the metallo-β-lactamase enzyme group (NMD-1 variant) in a sample (e.g. detecting, identifying, and/or analyzing the presence or absence of a transferrable genetic cassette harboring the NDM-1 variant), comprising: contacting a sample with detection reagents; and detecting the NDM-1 variant nucleic acid. In some embodiments, detecting, identifying, or analyzing the NDM-1 variant of the metallo-β-lactamase enzyme group in a sample comprises: contacting a sample with reagents for performing nucleic acid amplification (e.g., LATE-PCR); amplifying NMD-1 variant nucleic acid from the sample to generate amplified NMD-1 nucleic acid; and detecting the amplified NMD-1 nucleic acid. In some embodiments, kits are provided for detecting, identifying, or analyzing the NDM-1 variant in a sample, comprising: reagents for performing amplification (e.g., LATE-PCR) on NMD-1 variant nucleic acid. In some embodiments, the sample comprises an environmental sample. In some embodiments, the environmental sample is a water, soil, or food sample. In some embodiments, the sample is biological sample. In some embodiments, the biological sample is taken from a human, non-human primate, mammal, or animal. In some embodiments, the biological sample is a tissue sample. In some embodiments, the biological sample is a fluid sample (e.g. blood, saliva, urine). In some embodiments, the sample comprises a mixture of biological samples from multiple organisms. In some embodiments, NMD-1 variant nucleic acid is purified from the sample prior to amplification. In some embodiments, the sample contains less than 1000 copies of NMD-1 variant nucleic acid (e.g. less than 100 copies, less than 10 copies, etc.). In some embodiments, the reagents comprise amplification primers. In some embodiments, the amplification primers hybridize to the NMD-1 variant. In some embodiments, amplification primers comprise one or more of: ACAGCCTGACTTTCGCCGCC (SEQ ID NO: 3), AAGCGATGTCGGTGCCGTCG (SEQ ID NO: 4), CGACGGCACCGACATCGCTT (SEQ ID NO:5), and SGCRCGSGCSSWSGCSGCRW (SEQ ID NO: 6), wherein R=A or G, W=A or T, and S=C or G. In some embodiments, one or more of the amplification primers hybridize to a region of the NMD-1 variant that differs from wild-type members of the metallo-β-lactamase enzyme group. In some embodiments, one or more of the amplification primers hybridize to a region of the NMD-1 variant that differs from one or more (e.g. all) other members of the metallo-β-lactamase enzyme group (e.g. bla_{IMP}, bla_{GIM}, bla_{GIM}, bla_{SIM}, bla_{KMH}, etc.). In some embodiments, primers hybridize preferentially to NDM-1 variant nucleic acid. In some embodiments, primers hybridize preferentially to non-NDM-1 variant nucleic acid. In some embodiments, primers preferentially hybridize to a region of NDM-1 variant or non-NDM-1 variant nucleic acid, but are non-extendable (e.g. non-complementary base at 3′ end). In some embodiments, the amplification primers comprise a limiting primer and an excess primer, wherein the limiting primer at its initial concentration has a melting temperature relative to a target sequence that is higher than or equal to the excess primer melting temperature relative to a the target sequence at its initial concentration, in accord with the teaching and theory of LATE-PCR. In some embodiments, the limiting primer comprises ACAGCCTGACTTTCGCCGCC (SEQ ID NO: 3), or a sequence having at least 70% identity therewith (e.g., greater than 80%, 90%, 95%, 98%, 99%). In some embodiments, the limiting primer comprises AAGCGATGTCGGTGCCGTCG (SEQ ID NO: 4), or a sequence having at least 70% identity therewith (e.g., greater than 80%, 90%, 95%, 98%, 99%). In some embodiments, the limiting primer comprises CGACGGCACCGACATCGCTT (SEQ ID NO:5), or a sequence having at least 70% identity therewith (e.g., greater than 80%, 90%, 95%, 98%, 99%). In some embodiments, the limiting primer comprises SGCRCGSGCSSWSGCSGCRW (SEQ ID NO: 6), wherein R=A or G, W=A or T, and S=C or G, or a sequence having at least 70% identity therewith (e.g., greater than 80%, 90%, 95%, 98%, 99%). In some embodiments, the excess primer comprises ACAGCCTGACTTTCGCCGCC (SEQ ID NO: 3), or a sequence having at least 70% identity therewith (e.g., greater than 80%, 90%, 95%, 98%, 99%). In some embodiments, the excess primer comprises AAGCGATGTCGGTGCCGTCG (SEQ ID NO: 4), or a sequence having at least 70% identity therewith (e.g., greater than 80%, 90%, 95%, 98%, 99%).

In some embodiments, the excess primer comprises CGACGGCACCGACATCGCTT (SEQ ID NO:5), or a sequence having at least 70% identity therewith (e.g., greater than 80%, 90%, 95%, 98%, 99%). In some embodiments, the excess primer comprises SGCRCGSGCSSWSGCSGCRW (SEQ ID NO: 6), wherein R=A or G, W=A or T, and S=C or G, or a sequence having at least 70% identity therewith (e.g., greater than 80%, 90%, 95%, 98%, 99%).

In some embodiments, the reagents comprise a probe. In some embodiments, the probe is a molecular beacon. In some embodiments, the probe comprises a fluorescent label. In some embodiments, the probe has a melting temperature relative to a target nucleic acid that is lower than the melting temperature of an annealing step in an amplification reaction used in the amplifying. In some embodiments, the probe melting temperature is approximately 55° C. or lower. In some embodiments, the probe comprises a sequence which hybridizes to amplified NMD-1 variant nucleic acid, or a sequence having at least 70% identity therewith (e.g., greater than 80%, 90%, 95%, 98%, 99%). In some embodiments, a probe hybridizes preferentially to the NDM-1 variant over non-NDM-1 nucleic acids. In some embodiments, a probe hybridizes preferentially to one or more non-NDM-1 nucleic acids (e.g. other non-NDM-1 bla genes) over NDM-1 variant nucleic acids. In some embodiments, preferential probe hybridization is sufficient to discriminate between NDM-1 variant nucleic acid and non-NDM-1 nucleic acid.

In some embodiments, the reagents comprise an internal control target sequence. In some embodiments, the internal control target sequence is not homologous to a NMD-1 variant sequence. In some embodiments, the detecting comprises determining an amount of NMD-1 variant nucleic acid in the sample. In some embodiments, the detecting comprises detecting fluorescence associate with binding of a probe to the amplified NMD-1 variant nucleic acid after amplifying is completed. In some embodiments, the detecting comprises conducting a melt curve analysis between a probe and the amplified target nucleic acid. In some embodiments, the detecting differentiates NMD-1 variant from one or more or all of wild-type members of the metallo-β-lactamase enzyme group, wild-type metallo-β-lactamase enzyme, VIM-1 variant metallo-β-lactamase, VIM-2 variant metallo-β-lactamase, etc. In some embodiments, detecting NMD-1 variant nucleic acid differentiates NMD-1 variant bacteria from other non-NMD-1 variant bacteria. In some embodiments, the detecting NMD-1 variant nucleic acid differentiates NMD-1 variant bacteria from other antibiotic resistant bacteria. In some embodiments, the detecting NMD-1 variant nucleic acid differentiates NMD-1 variant bacteria from other multiple antibiotic resistant bacteria. In some embodiments, the reagents comprise Primesafe™II. In some embodiments, detecting NMD-1 variant nucleic acid identifies the strain of NMD-1 variant bacteria. In some embodiments, the reagents are contained within a reaction cartridge. In some embodiments, the reaction cartridge is configured to interact with a portable sample preparation and PCR instrument. In some embodiments, the portable sample preparation and PCR instrument comprises the BIO-SEEQ (Smiths Detection Inc., Edgewood, Md.) Portable Veterinary Diagnostics Laboratory.

### BRIEF DESCRIPTION OF THE DRAWINGS

FIG. 1 shows an illustration of an exemplary primer scheme for detection of NDM-1 variant through primer binding to the insert.

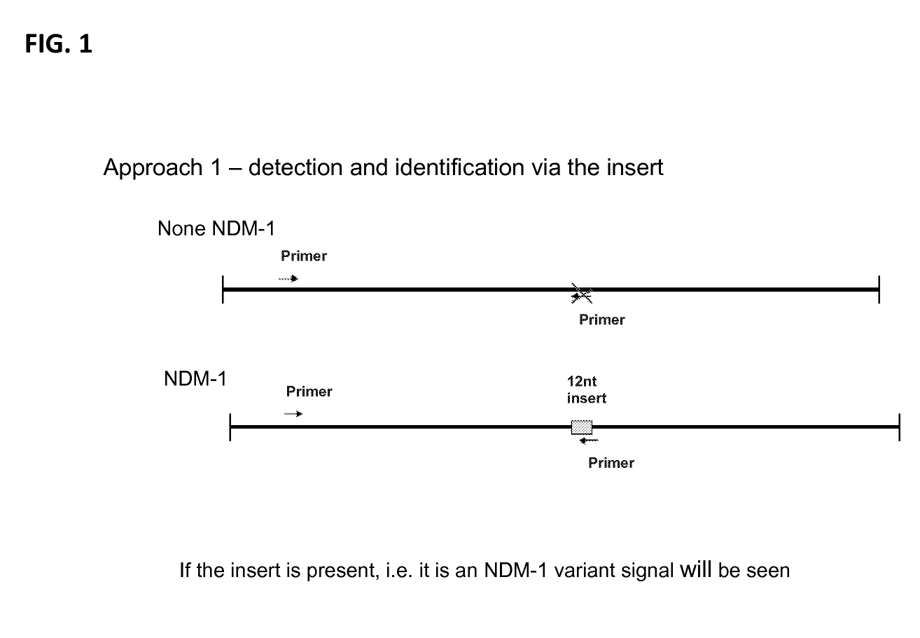

FIG. 2 shows an illustration of an exemplary primer scheme for detection of NDM-1 variant through lack of primer binding to the insert.

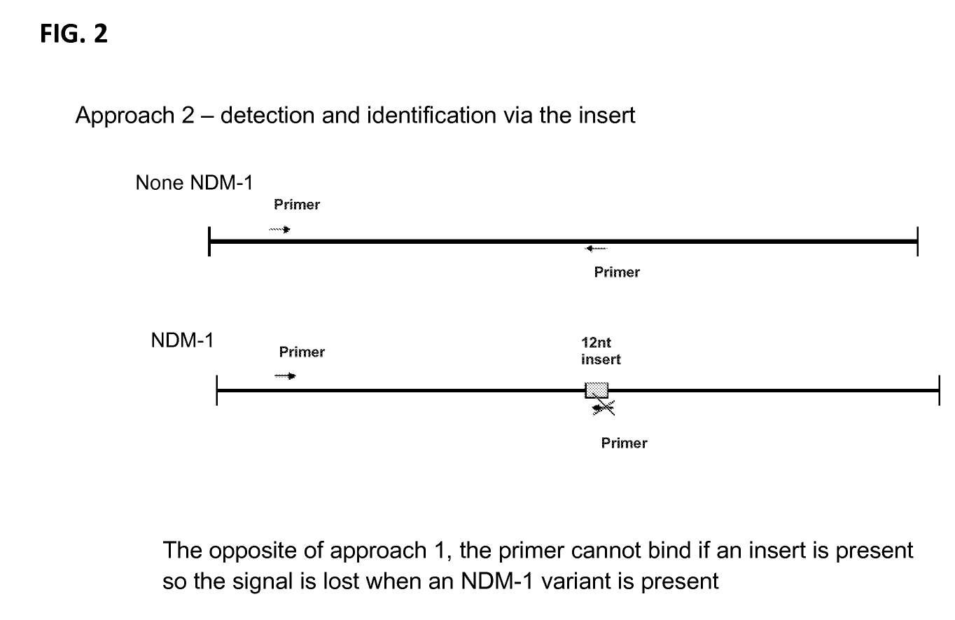

FIG. 3 shows an illustration of an exemplary primer design for detection of NDM-1 variant through primer binding to the SNP.

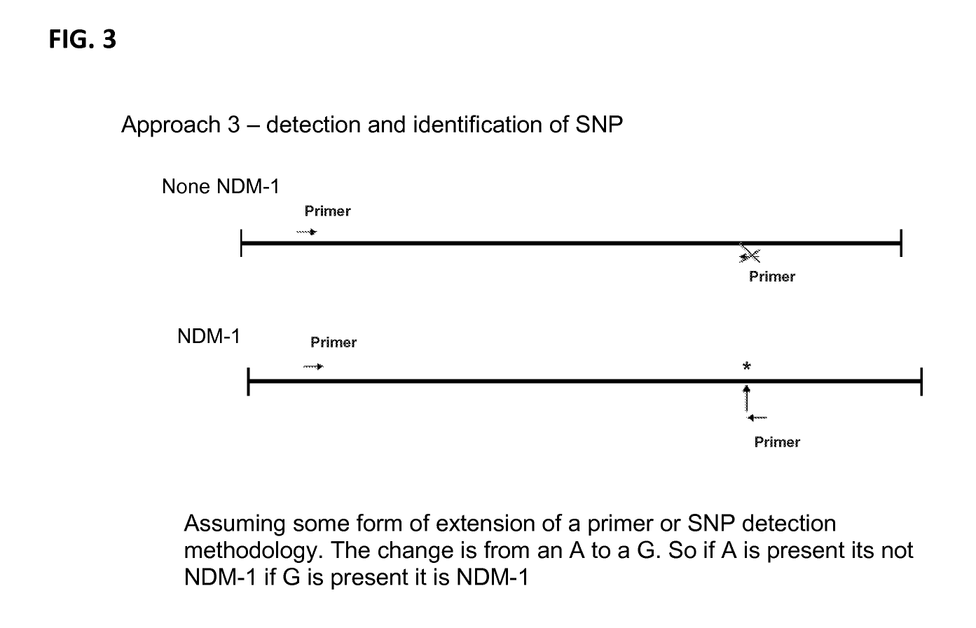

FIG. 4 shows an illustration of an exemplary primer design for detection of NDM-1 variant through lack of primer binding to the SNP.

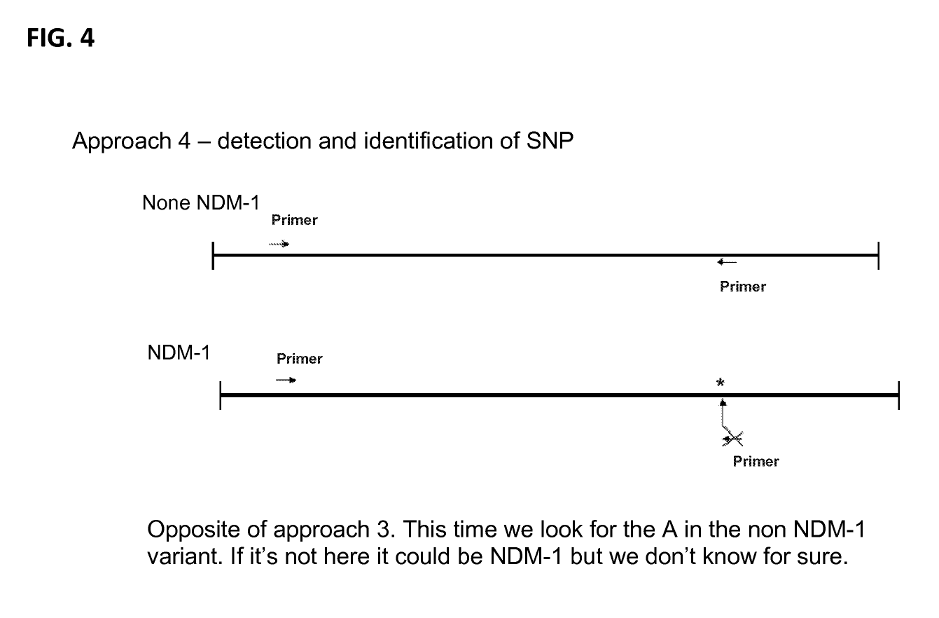

### DEFINITIONS

As used herein, the term “NMD-1 variant” refers to a NDM-1 variant of the metallo-β-lactamase enzyme. Unless otherwise specified, “NMD-1 variant” may refer to the “MBL_{NDM-1}” gene, a nucleic acid (NMD-1 variant nucleic acid) capable of expressing a NDM-1 variant of the metallo-β-lactamase enzyme (e.g. bla_{NDM-1}, bla_{NDM-1} and surrounding nucleic acid), a plasmid (NMD-1 variant plasmid) carrying the NDM-1 variant gene, the NDM-1 variant of the metallo-β-lactamase enzyme (NDM-1 variant enzyme), or a bacteria (NDM-1 variant bacteria) expressing and/or harboring a NDM-1 variant of the metallo-β-lactamase enzyme group.

As used herein, the term “molecular beacon probe” refers to a single-stranded oligonucleotide, typically 25 to 35 bases-long, in which the bases on the 3′ and 5′ ends are complementary forming a “stem,” typically for 5 to 8 base pairs. In certain embodiments, the molecular beacons employed have stems that are exactly 2 or 3 base pairs in length. A molecular beacon probe forms a hairpin structure at temperatures at and below those used to anneal the primers to the template (typically below about 60° C.). The double-helical stem of the hairpin brings a fluorophore (or other label) attached to the 5′ end of the probe very close to a quencher attached to the 3′ end of the probe. The probe does not fluoresce (or otherwise provide a signal) in this conformation. If a probe is heated above the temperature used to melt the double stranded stem apart, or the probe is allowed to hybridize to a target oligonucleotide that is complementary to the sequence within the single-strand loop of the probe, the fluorophore and the quencher are separated, and the fluorophore fluoresces in the resulting conformation. Therefore, in a series of

PCR cycles the strength of the fluorescent signal increases in proportion to the amount of the beacon hybridized to the amplicon, when the signal is read at the annealing temperature. Molecular beacons with different loop sequences can be conjugated to different fluorophores in order to monitor increases in amplicons that differ by as little as one base (Tyagi, S. and Kramer, F. R. (1996), Nat. Biotech. 14:303 308; Tyagi, S. et al., (1998), Nat. Biotech. 16: 49 53; Kostrikis, L. G. et al., (1998), Science 279: 1228 1229; all of which are herein incorporated by reference).

As used herein, the term “amplicon” refers to a nucleic acid generated using primer pairs, such as those described herein. The amplicon may be single-stranded DNA (e.g., the result of asymmetric amplification) or double stranded DNA, however, it may be RNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. In certain embodiments, the type of amplification is asymmetric PCR (e.g., LATE-PCR) which is described in, for example, U.S. Pat. No. 7,198,897 and Pierce et al., PNAS, 2005, 102(24):8609-8614, both of which are herein incorporated by reference in their entireties.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The terms “homology,” “homologous,” and “sequence identity” refer to a degree of identity. There may be partial homology or complete homology. A partially homologous sequence is one that is less than 100% identical to another sequence. Determination of sequence identity is described in the following example: a primer 20 nucleobases in length which is otherwise identical to another 20 nucleobase primer but having two non-identical residues has 18 of 20 identical residues (18/20=0.9 or 90% sequence identity). In another example, a primer 15 nucleobases in length having all residues identical to a 15 nucleobase segment of a primer 20 nucleobases in length would have 15/15=1.0 or 100% sequence identity with 75% of the 20 nucleobase primer. Sequence identity may also encompass alternate or “modified” nucleobases that perform in a functionally similar manner to the regular nucleobases adenine, thymine, guanine and cytosine with respect to hybridization and primer extension in amplification reactions. In a non-limiting example, if the 5-propynyl pyrimidines propyne C and/or propyne T replace one or more C or T residues in one primer which is otherwise identical to another primer in sequence and length, the two primers will have 100% sequence identity with each other. In another non-limiting example, Inosine (I) may be used as a replacement for G or T and effectively hybridize to C, A or U (uracil). Thus, if inosine replaces one or more C, A or U residues in one primer which is otherwise identical to another primer in sequence and length, the two primers will have 100% sequence identity with each other. Other such modified or universal bases may exist which would perform in a functionally similar manner for hybridization and amplification reactions and will be understood to fall within this definition of sequence identity.

As used herein, the term “hybridization” or “hybridize” is used in reference to the pairing of complementary nucleic acids. The strength of hybridization is expressed by the melting temperature, or effective melting temperature of hybridized nucleic acids. Melting temperature is influenced by such factors as the degree of complementary between the nucleic acids, stringency of the conditions involved, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference.

As used herein, the term “primer” refers to an oligonucleotide with a 3′OH, whether occurring naturally as in a purified restriction digest or produced synthetically, that is capable of forming a short double-stranded DNA/DNA or DNA/RNA hybrid on a longer template strand for initiation of synthesis via primer extension under permissive conditions (e.g., in the presence of nucleotides and an inducing agent such as a biocatalyst (e.g., a DNA polymerase or the like) and at a suitable temperature, pH, and ion composition). The primer is typically single stranded for maximum efficiency in amplification, but may alternatively be double stranded or partially double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

In some embodiments, the oligonucleotide primer pairs described herein can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein, the term “nucleic acid molecule” refers to any nucleic acid containing molecule, including but not limited to, DNA or RNA. The term encompasses sequences that include any of the known base analogs of DNA and RNA including, but not limited to, 4 acetylcytosine, 8-hydroxy-N6-methyladenosine, aziridinylcytosine, pseudoisocytosine, 5-(carboxyhydroxyl-methyl)uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68: 90-99; the phosphodiester method of Brown et al. (1979) Meth Enzymol. 68: 109-151; the diethylphosphoramidite method of Beaucage et al. (1981) Tetrahedron Lett. 22: 1859-1862; the triester method of Matteucci et al. (1981) J Am Chem Soc. 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein a “sample” refers to anything capable of being analyzed by the methods provided herein. In some embodiments, the sample comprises or is suspected to comprise one or more nucleic acids capable of analysis by the methods. Preferably, the samples comprise nucleic acids (e.g., DNA, RNA, cDNAs, etc.) from one or more bioagents, such as bacteria harboring an NDM-1 variant nucleic acid. Samples can include, for example, blood, saliva, urine, feces, anorectal swabs, vaginal swabs, cervical swabs, nasal swabs, and the like. Sample may also be environmental samples, such as soil, water, and the like. A sample may also comprise an agricultural product, such as meat, fruit, vegetables, dairy, eggs, bread, etc. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

### DETAILED DESCRIPTION OF THE INVENTION

Herein are provided compositions and methods for the detection, identification, and analysis of NDM-1 variant nucleic acids and bacteria, or other organisms harboring such sequences. In particular, the kits, compositions, and methods herein employ amplification reagents and processes for the detection, identification, and analysis of NDM-1 variant nucleic acids and bacteria. In some embodiments, the kits, compositions, and methods employ LATE-PCR reagents and processes for the detection and analysis of NDM-1 variant nucleic acids and bacteria, although the embodiments are not limited to LATE-PCR. In some embodiments, kits, compositions, and methods employ single probe, multiple temperature, nucleic acid detection methods (See, e.g. International Application No. PCT/US10/45199, herein incorporated by reference in its entirety).

Mobile class B β-lactamases, or metallo-β-lactamases (MBLs) are able to hydrolyze penicillins, cephalosporins, and carbapenems, thereby conferring antibiotic resistance to a broad set of important antibiotics. MBLs are most commonly found in Pseudomonas aeruginosa, but NDM-1 is increasingly being found in Enterobacteriaceace, including Escherichia coli and Klebsiella pneumonia. NDM-1 (New Delhi metallo-β-lactamase) is a variant of the metallo-β-lactamase group which provides highly effective and broad antibiotic resistance to bacteria which harbot it. NDM-1 displays tighter binding to most cephalosporins and penicillins than other class B β-lactamases. The NDM-1 variant shares little identity with other MBLs. It is most similar to MBLs VIM-1 and VIM-2, with which it has 32.4% identity. In some embodiments, the NDM-1 variant metallo-β-lactamase enzyme is encoded by the nucleotide sequence according to GenBank accession number AB571289:

| (SEQ ID NO: 1) | |
|---|---|
| 61 | ccgcgtgctg gtggtcgata ccgcctggac cgatgaccag accgcccaga tcctcaactg |
| 121 | gatcaagcag gagatcaacc tgccggtcgc gctggcggtg gtgactcacg cgcatcagga |
| 181 | caagatgggc ggtatggacg cgctgcatgc ggcggggatt gcgacttatg ccaatgcgtt |
| 241 | gtcgaaccag cttgccccgc aagaggggat ggttgcggcg caacacagcc tgactttcgc |
| 301 | cgccaatggc tgggtcgaac cagcaaccgc gcccaacttt ggcccgctca aggtatttta |
| 361 | ccccggcccc ggccacacca gtgacaatat caccgttggg atcgacggca ccgacatcgc |
| 421 | ttttggtggc tgcctgatca aggacagcaa ggccaagtcg ctcggcaatc tcggt. |

In comparison to MBLs VIM-1 and VIM-2, NDM-1 comprises a 4 amino acid insert of Phe-Ala-Ala-Asn (SEQ ID NO: 7) between positions 162 and 166, and 2 amino acid substitutions at positions 116 and 118 (Yong et al. antimicrobial Agents and Chemotherapy. December 2009, p. 5046-5054., herein incorporated by reference in its entirety). In some embodiments, the 4 amino acid insert (a.k.a. NMD-1 insertion) is encoded by the nucleotide sequence TTCGCCGCCAAT (SEQ ID NO:2). In some embodiments, the bla_{NDM-1} gene, which codes for the NDM-21 variant enzyme is carried on a plasmid (NDM-1 variant plasmid). In some embodiments, NDM-1 variant plasmid comprises other antibiotic resistance genes (e.g. ereC), other proteins, transcription and translation regulatory elements, etc. In some embodiments, a NDM-1 variant nucleic acid comprises a metallo-β-lactamase gene variant (bla_{NDM-1}) and a truncated IS26 element.

In some embodiments, the NDM-1 variant nucleic acid comprises a nucleic acid (e.g. plasmid) which confers resistance to a wide range of antibiotics. In some embodiments, a wide range of antibiotic resistance (e.g. penicillin class antibiotics, cephalosporin class antibiotics, and carbapenem class antibiotics) is conferred to the bacteria harboring the NDM-1 variant nucleic acid. In some embodiments, the NDM-1 variant nucleic acid comprises a plasmid capable of transfer from one bacterium to another. In some embodiments, the NDM-1 variant nucleic acid comprises a plasmid capable of transfer from one bacterial strain to another. In some embodiments, the NDM-1 variant nucleic acid comprises a plasmid capable of transfer from one bacterial species to another. In some embodiments, a NDM-1 variant plasmid is any plasmid which comprises the NDM-1 variant gene. A NDM-1 variant plasmid comprises the bla_{NDM-1} gene and any other nucleic acids suitable for plasmid formation and/or transfer. In some embodiments, the compositions, methods, and kits are provided to identify nucleic acid sequences characteristic of NMD-1 variant nucleic acids (e.g. NMD-1 insertion sequence (SEQ ID NO: 2), NMD-1 SNPs, etc.). In some embodiments, compositions, methods, and kits are provided to identify nucleic acid sequences comprising nucleic acid sequences characteristic of NMD-1 variant nucleic acids (e.g. NMD-1 insertion sequence (SEQ ID NO: 2), NMD-1 SNPs, etc.). In some embodiments, the compositions, methods, and kits are provided to identify transferable elements, plasmids, cassettes, integrons, and/or genetic elements comprising one or more nucleic acid sequences characteristic of NMD-1 variant nucleic acids (e.g. NMD-1 insertion sequence (SEQ ID NO: 2), NMD-1 SNPs, etc.). In some embodiments, transferable elements, plasmids, cassettes, integrons, and/or genetic elements comprising one or more nucleic acid sequences characteristic of NMD-1 variant nucleic acids further comprise additional nucleic acid sequences, for example coding for other proteins, antibiotic resistance genes, and/or transcription and translation regulatory elements. In some embodiments, the compositions, methods, and kits are provided to identify bacteria or other organisms harboring NMD-1 variant nucleic acid sequences or one or more nucleic acid sequences characteristic of NMD-1 variant nucleic acids (e.g. NMD-1 insertion sequence (SEQ ID NO: 2), NMD-1 SNPs, etc.).

In some embodiments, compositions (e.g. primers, probes, reagents, etc.), methods, and kits are provided to identify nucleic acids (e.g. genomic, plasmid, synthetic, etc.) comprising the NDM-1 variant nucleic acid, and plasmids or bacteria expressing and/or harboring NDM-1 variant nucleic acid. In some embodiments, methods and kits herein provide detection, identification, and/or quantification of nucleic acids comprising NDM-1 nucleic acid. In some embodiments, methods and kits herein provide detection, identification, and/or quantification of nucleic acids comprising, consisting essentially of, or consisting of NDM-1 nucleic acid (e.g. bla_{NDM-1}). In some embodiments, methods and kits provide detection, identification, and/or quantification of nucleic acids comprising the complete bla_{NDM-1} gene (e.g. SEQ ID NO:1). In some embodiments, methods and kits provide detection, identification, and/or quantification of nucleic acids comprising a portion of the bla_{NDM-1} gene. In some embodiments, methods and kits provide detection, identification, and/or quantification a portion of NMD-1 variant nucleic acid. In some embodiments, methods and kits provide detection, identification, and/or quantification of nucleic acids comprising the region of bla_{NDM-1} surrounding the NMD-1 insertion (e.g. SEQ ID NO: 2). In some embodiments, methods and kits provide detection, identification, and/or quantification of nucleic acids comprising the region of bla_{NDM-1} surrounding SEQ ID NO: 2 (e.g. 10 or more nucleotides from bla_{NDM-1} surrounding SEQ ID NO: 2, 20 or more nucleotides from bla_{NDM-1} surrounding SEQ ID NO: 2, 30 or more nucleotides from bla_{NDM-1} surrounding SEQ ID NO: 2, 40 or more nucleotides from bla_{NDM-1} surrounding SEQ ID NO: 2, 50 or more nucleotides from bla_{NDM-1} surrounding SEQ ID NO: 2, etc). In some embodiments, methods and kits provide detection, identification, and/or quantification of nucleic acid comprising a region of the bla_{NDM-1} gene (e.g. 6 nucleotides, 10 nucleotides, 20 nucleotides, 30 nucleotides, 40 nucleotides, 50 nucleotides, etc.) surrounding SEQ ID NO:2. In some embodiments, methods and kits provide detection, identification, and/or quantification of nucleic acids comprising the region of bla_{NDM-1} sequence encoding SEQ ID NO: 7 (e.g. 10 or more nucleotides surrounding the region encoding SEQ ID NO: 7, 20 or more nucleotides surrounding the region encoding SEQ ID NO: 7, 30 or more nucleotides surrounding the region encoding SEQ ID NO: 7, 40 or more nucleotides surrounding the region encoding SEQ ID NO: 7, 50 or more nucleotides surrounding the region encoding SEQ ID NO: 7, etc). In some embodiments, methods and kits provide detection, identification, and/or quantification of nucleic acid comprising a region of the bla_{NDM-1} gene (e.g. 6 nucleotides, 10 nucleotides, 20 nucleotides, 30 nucleotides, 40 nucleotides, 50 nucleotides, etc.) surrounding the sequence coding for SEQ ID NO:7. In some embodiments, methods and kits provide detection, identification, and/or quantification of nucleic acids comprising a portion of the bla_{NDM-1} gene surrounding the NDM-1 substitutions at positions 116 and 118 (aka. NMD-1 single nucleotide polymorphisms (SNPs). In some embodiments, methods and kits provide detection, identification, and/or quantification of nucleic acids comprising a portion of the bla_{NDM-1} gene surrounding the NDM-1 substitutions at positions 116 and 118 (e.g. 10 or more nucleotides from bla_{NDM-1} surrounding 116-118, 20 or more nucleotides from bla_{NDM-1} surrounding 116-118, 30 or more nucleotides from bla_{NDM-1} surrounding 116-118, 40 or more nucleotides from bla_{NDM-1} surrounding 116-118, 50 or more nucleotides from bla_{NDM-1} surrounding 116-118, etc). In some embodiments, methods and kits provide detection, identification, and/or quantification of nucleic acid comprising a region of the bla_{NDM-1} gene (e.g. 6 nucleotides, 10 nucleotides, 20 nucleotides, 30 nucleotides, 40 nucleotides, 50 nucleotides, etc.) surrounding the NDM-1 substitutions at positions 116 and 118. In some embodiments, kits and methods provide detection, identification, and/or quantification of nucleic acid comprising the NDM-1 insertion of SEQ ID NO: 2 and NDM-1 substitutions at positions 116 and 118. In some embodiments, kits and methods provide detection, identification, and/or quantification of nucleic acid comprising the sequence coding for the NDM-1 insertion of SEQ ID NO:7, and NDM-1 substitutions at positions 116 and 118.

In some embodiments, compositions, methods, and kits further provide detecting, detection, identification, and/or quantification of one or more additional genes and/or nucleic acid segments (e.g. additional resistance genes, additional mutations, sequence variations, etc.). In some embodiments, reagents (e.g. primers, probes, etc.) are provided for detection of NDM-1 nucleic acid and one or more of: antibiotic resistance genes, bacterial species markers, bacterial strain markers, markers of the context of bla_{NDM-1}, etc.

In certain embodiments, the assays described herein employ primer pairs to amplify target nucleic acid sequences. The methods described herein are not limited by the type of amplification that is employed. In certain embodiments, PCR, asymmetric PCR, and/or LATE-PCR, is employed.

PCR is a repeated series of steps of denaturation, or strand melting, to create single-stranded templates; primer annealing; and primer extension by a thermally stable DNA polymerase such as Thermus aquaticus (Taq) DNA polymerase. A typical three-step PCR protocol (see Innis et al., Chapter 1) may include denaturation, or strand melting, at 93-95 degrees C. for more than 5 sec, primer annealing at 55-65 degrees C. for 10-60 sec, and primer extension for 15-120 sec at a temperature at which the polymerase is highly active, for example, 72 degrees C. for Taq DNA polymerase. A typical two-step PCR protocol may differ by having the same temperature for primer annealing as for primer extension, for example, 60 degrees C. or 72 degrees C. For either three-step PCR or two-step PCR, an amplification involves cycling the reaction mixture through the foregoing series of steps numerous times, typically 25-40 times. During the course of the reaction the times and temperatures of individual steps in the reaction may remain unchanged from cycle to cycle, or they may be changed at one or more points in the course of the reaction to promote efficiency or enhance selectivity. In addition to the pair of primers and target nucleic acid a PCR reaction mixture typically contains each of the four deoxyribonucleotide 5′ triphosphates (dNTPs) at equimolar concentrations, a thermostable polymerase, a divalent cation, and a buffering agent. A reverse transcriptase is included for RNA targets, unless the polymerase possesses that activity. The volume of such reactions is typically 25-100 ul. Multiple target sequences can be amplified in the same reaction. In the case of cDNA amplification, PCR is preceded by a separate reaction for reverse transcription of RNA into cDNA, unless the polymerase used in the PCR possesses reverse transcriptase activity. The number of cycles for a particular PCR amplification depends on several factors including: a) the amount of the starting material, b) the efficiency of the reaction, and c) the method and sensitivity of detection or subsequent analysis of the product. Cycling conditions, reagent concentrations, primer design, and appropriate apparatuses for typical cyclic amplification reactions are well known in the art.

Ideally, each strand of each amplicon molecule binds a primer at one end and serves as a template for a subsequent round of synthesis. The rate of generation of primer extension products, or amplicons, is thus generally exponential, theoretically doubling during each cycle. The amplicons include both plus (+) and minus (−) strands, which hybridize to one another to form double strands. To differentiate typical PCR from special variations described herein, typical PCR is referred to as “symmetric” PCR. Symmetric PCR thus results in an exponential increase of one or more double-stranded amplicon molecules, and both strands of each amplicon accumulate in equal amounts during each round of replication. The efficiency of exponential amplification via symmetric PCR eventually declines, and the rate of amplicon accumulation slows down and stops. Kinetic analysis of symmetric PCR reveals that reactions are composed of: a) an undetected amplification phase (initial cycles) during which both strands of the target sequence increase exponentially, but the amount of the product thus far accumulated is below the detectable level for the particular method of detection in use; b) a detected amplification phase (additional cycles) during which both strands of the target sequence continue to increase in parallel and the amount of the product is detectable; c) a plateau phase (terminal cycles) during which synthesis of both strands of the amplicon gradually stops and the amount of product no longer increases. Symmetric reactions slow down and stop because the increasing concentrations of complementary amplicon strands hybridize to each other (reanneal), and this out-competes the ability of the separate primers to hybridize to their respective target strands. Typically reactions are run long enough to guarantee accumulation of a detectable amount of product, without regard to the exact number of cycles needed to accomplish that purpose.

A technique that has found limited use for making single-stranded DNA directly in a PCR reaction is “asymmetric PCR.” Gyllensten and Erlich, “Generation of Single-Stranded DNA by the polymerase chain reaction and its application to direct sequencing of the HLA-DQA Locus,” Proc. Natl. Acad. Sci. (USA) 85: 7652 7656 (1988); Gyllensten, U. B. and Erlich, H. A. (1991) “Methods for generating single stranded DNA by the polymerase chain reaction” U.S. Pat. No. 5,066,584, Nov. 19, 1991; all of which are herein incorporated by reference. Asymmetric PCR differs from symmetric PCR in that one of the primers is added in limiting amount, typically 1/100th to ⅕th of the concentration of the other primer. Double-stranded amplicon accumulates during the early temperature cycles, as in symmetric PCR, but one primer is depleted, typically after 15-25 PCR cycles, depending on the number of starting templates. Linear amplification of one strand takes place during subsequent cycles utilizing the undepleted primer. Primers used in asymmetric PCR reactions reported in the literature, including the Gyllensten patent, are often the same primers known for use in symmetric PCR. Poddar (Poddar, S. (2000) “Symmetric vs. Asymmetric PCR and Molecular Beacon Probe in the Detection of a Target Gene of Adenovirus,” Mol. Cell Probes 14: 25 32 compared symmetric and asymmetric PCR for amplifying an adenovirus substrate by an end-point assay that included 40 thermal cycles. He reported that a primers ratio of 50:1 was optimal and that asymmetric PCR assays had better sensitivity that, however, dropped significantly for dilute substrate solutions that presumably contained lower numbers of target molecules. In some embodiments, asymmetric PCR is used with embodiments of the assays described herein.

In some embodiments, kits, compositions, and methods for NDM-1 variant detection are based on Linear-After-The-Exponential (LATE) PCR (Pierce et al. Methods Mol Med. 2007; 132:65-85., herein incorporated by reference in its entirety), an advanced form of asymmetric PCR, that allows for rapid and sensitive detection at endpoint, together with Primesafe™II (Rice et al. Nat Protoc. 2007;2(10):2429-38., herein incorporated by reference in its entirety), a PCR additive that maintains the fidelity of amplification over a broad range of target concentrations by suppressing mis-priming throughout the reaction. LATE-PCR assays reliably generate abundant single-stranded amplicons that can readily be detected in real-time and/or characterized at end-point using probes. In some embodiments, the assay functions as a duplex with an internal DNA control. The LATE-PCR assay described here can be used on both standard laboratory equipment or in the BIO-SEEQ Portable Veterinary Diagnostics Laboratory, a portable sample preparation and PCR instrument built by Smiths Detection. This device is specifically engineered for use in the field with a minimum of operator training It includes an automated sample preparation unit that carries out sample preparation and LATE-PCR analysis on site in a matter of hours. Individual sample preparation units for the BIO-SEEQ II, as well as the entire machine can be immersed in disinfectants (Virkon or Fam30) so as to ensure that contaminants (e.g. bacteria) is not transported away from the site of field testing.

Linear-After-The-Exponential-PCR (LATE-PCR) is an advanced form of asymmetric PCR. By applying this principle, a powerful assay for NDM-1 variant detection and identification is provided. The LATE-PCR assay is capable of detecting below 10 copies of a nucleic acid in clinical specimens. Since the assay is designed to be used in either laboratory settings or in a portable PCR machine (BIO-SEEQ Portable Veterinary Diagnostics Laboratory; Smiths Detection, Watford UK), the LATE-PCR provides a robust tool for the detection, identification, and analysis of NMD-1 variants, both in diagnostic institutes and in the field.

When using LATE-PCR, each reaction produces large amounts of specific, single-stranded DNA, which can then be probed with a sequence-specific probe. When tested against synthetic targets, the assay proved to be specific and effective even at low target numbers. Indeed, this assay generated robust specific signals down to approximately 1 molecule/reaction. The internal DNA control present in the assay is also specific and sensitive at low copy number.

LATE-PCR includes innovations in primer design, in temperature cycling profiles, and in hybridization probe design. Being a type of PCR process, LATE-PCR utilizes the basic steps of strand melting, primer annealing, and primer extension by a DNA polymerase caused or enabled to occur repeatedly by a series of temperature cycles. In the early cycles of a LATE-PCR amplification, when both primers are present, LATE-PCR amplification amplifies both strands of a target sequence exponentially, as occurs in conventional symmetric PCR. LATE-PCR then switches to synthesis of only one strand of the target sequence for additional cycles of amplification. In certain real-time LATE-PCR assays, the limiting primer is exhausted within a few cycles after the reaction reaches its C_{T} value, and in the certain assays one cycle after the reaction reaches its C_{T} value. As defined above, the C_{T} value is the thermal cycle at which signal becomes detectable above the empirically determined background level of the reaction. Whereas a symmetric PCR amplification typically reaches a plateau phase and stops generating new amplicons by the 50th thermal cycle, LATE-PCR amplifications do not plateau, because the do not continue to accumulate double-stranded products, and thus continue to generate single-stranded amplicons well beyond the 50th cycle, even through the 100th cycle. LATE-PCR amplifications and assays typically include at least 60 cycles, preferably at least 70 cycles when small (10,000 or less) numbers of target molecules are present at the start of amplification.

With certain exceptions, the ingredients of a reaction mixture for LATE-PCR amplification are generally the same as the ingredients of a reaction mixture for a corresponding symmetric PCR amplification. The mixture typically includes each of the four deoxyribonucleotide 5′ triphosphates (dNTPs) at equimolar concentrations, a thermostable polymerase, a divalent cation, and a buffering agent. As with symmetric PCR amplifications, it may include additional ingredients, for example reverse transcriptase for RNA targets. Non-natural dNTPs may be utilized. For instance, dUTP can be substituted for dTTP and used at 3 times the concentration of the other dNTPs due to the less efficient incorporation by Taq DNA polymerase.

In certain embodiments, the starting molar concentration of one primer, the “Limiting Primer,” is less than the starting molar concentration of the other primer, the “Excess Primer.” The ratio of the starting concentrations of the Excess Primer and the Limiting Primer is generally at least 5:1, preferably at least 10:1, and more preferably at least 20:1. The ratio of Excess Primer to Limiting Primer can be, for example, 5:1 . . . 10:1, 15:1 . . . 20:1 . . . 25:1 . . . 30:1 . . . 35:1 . . . 40:1 . . . 45:1 . . . 50:1 . . . 55:1 . . . 60:1 . . . 65:1 . . . 70:1 . . . 75:1 . . . 80:1 . . . 85:1 . . . 90:1 . . . 95:1 . . . or 100:1 . . . 1000:1 . . . or more. Primer length and sequence are adjusted or modified, preferably at the 5′ end of the molecule, such that the concentration-adjusted melting temperature of the Limiting Primer at the start of the reaction, T_{M}[0]^{L}, is greater than or equal (plus or minus 0.5 degrees C.) to the concentration-adjusted melting point of the Excess Primer at the start of the reaction, T_{M}[0]^{X}. Preferably the difference (T_{M}[0]^{L}-T_{M}[0]^{X}) is at least +3, and more preferably the difference is at least +5 degrees C.

Amplifications and assays according to embodiments of methods described herein can be performed with initial reaction mixtures having ranges of concentrations of target molecules and primers. LATE-PCR assays are particularly suited for amplifications that utilize small reaction-mixture volumes and relatively few molecules containing the target sequence, sometimes referred to as “low copy number.” While LATE-PCR can be used to assay samples containing large amounts of target, for example up to 10⁶ copies of target molecules, other ranges that can be employed are much smaller amounts, from to 1-50,000 copies, 1-10,000 copies and 1-1,000 copies. In certain embodiments, the concentration of the Limiting Primer is from a few nanomolar (nM) up to 200 nM. The Limiting Primer concentration is preferably as far toward the low end of the range as detection sensitivity permits.

In some embodiments compositions (e.g., kits, kit components, systems, instruments, reaction mixtures) comprising one or more or all of the components useful, necessary, or sufficient for carrying out any of the methods described herein are provided. In some embodiments, kits are provided containing one or more or all of the reagents.

### Experimental

The following examples provide specific embodiments which find use with the present invention. These examples should be viewed as examples and not as limiting the scope of the invention.

### EXAMPLE 1

### NDM-1 Insertion Detection by PCR

In some embodiments, PCR amplification techniques are used to identify the presence or absence of nucleic acids encoding the NDM-1 variant of the metallo-β-lactamase enzyme group in a sample. A forward primer corresponding to ACAGCCTGACTTTCGCCGCC (SEQ ID NO: 3) and reverse primer corresponding to AAGCGATGTCGGTGCCGTCG (SEQ ID NO: 4) are used to amplify the region containing the 12 nucleotide insertion characteristic of the NDM-1 variant (SEQ ID NO: 2). Detection of amplification products corresponding to this region indicates the presence of nucleic acids encoding the NDM-1 variant of the metallo-β-lactamase enzyme group in the sample. The absence of amplification products corresponding to this region indicates the absence of nucleic acids encoding the NDM-1 variant of the metallo-β-lactamase enzyme group in the sample.

### EXAMPLE 2

### NDM-1 SNP Detection by PCR

In some embodiments, PCR amplification techniques are used to identify the presence or absence of nucleic acids encoding the NDM-1 variant of the metallo-β-lactamase enzyme group in a sample. A forward primer corresponding to CGACGGCACCGACATCGCTT (SEQ ID NO:5) and reverse primer corresponding to SGCRCGSGCSSWSGCSGCRW (SEQ ID NO: 6) are used to amplify the region containing the single nucleotide polymorphisms at positions 116 and 118 that are characteristic of the NDM-1 variant. Detection of amplification products corresponding to this region indicate the presence of nucleic acids encoding the NDM-1 variant of the metallo-β-lactamase enzyme group in the sample. The absence of amplification products corresponding to this region indicates the absence of nucleic acids encoding the NDM-1 variant of the metallo-β-lactamase enzyme group in the sample.

### EXAMPLE 3

### PCR Detection of NDM-1

In some embodiments, PCR amplification techniques are used to identify the presence or absence of nucleic acids encoding the NDM-1 variant of the metallo-β-lactamase enzyme group in a sample. Using two set of primers provides further confirmation of the presence of nucleic acid conferring the antibiotic resistance of the NDM-1 variant. A forward primer corresponding to ACAGCCTGACTTTCGCCGCC (SEQ ID NO: 3) and reverse primer corresponding to AAGCGATGTCGGTGCCGTCG (SEQ ID NO: 4) are used to amplify the region containing the 12 nucleotide insertion characteristic of the NDM-1 variant (SEQ ID NO: 2). Further, a forward primer corresponding to CGACGGCACCGACATCGCTT (SEQ ID NO: 5) and reverse primer corresponding to SGCRCGSGCSSWSGCSGCRW (SEQ ID NO: 6) are used to amplify the region containing the single nucleotide polymorphisms at positions 116 and 118 that are characteristic of the NDM-1 variant. Detection of both amplification products indicates the presence of nucleic acids encoding the NDM-1 variant of the metallo-β-lactamase enzyme group in the sample. The absence of amplification products corresponding to these regions indicates the absence of nucleic acids encoding the NDM-1 variant of the metallo-β-lactamase enzyme group in the sample.

Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method for detecting variant NMD-1 in a sample, comprising:
a) contacting a sample with NMD-1 variant detection reagents;
b) amplifying a region of NMD-1 variant nucleic acid comprising the NDM-1 insertion and/or NMD-1 SNP to generate amplified NMD-1 variant nucleic acid; and
c) detecting said amplified NMD-1 variant insert nucleic acid.

2. The method of claim 1, wherein NMD-1 variant detection reagents comprise NMD-1 amplification reagents.

3. The method of claim 2, wherein NMD-1 amplification reagents comprise reagents for performing LATE-PCR.

4. The method of claim 3, further comprising a step between step (a) and step (b) comprising: amplifying NMD-1 variant nucleic acid from said sample to generate amplified NMD-1 variant nucleic acid.

5. The method of claim 1, wherein said sample contains less than 10 copies of NMD-1 variant nucleic acid.

6. The method of claim 2, wherein said amplification reagents comprise amplification primers.

7. The method of claim 6, wherein said amplification primers hybridize to NMD-1 variant nucleic acid.

8. The method of claim 7, wherein said amplification primers comprise SEQ ID NOS: 3 and 4, or sequences having at least 70% identity therewith.

9. The method of claim 1, wherein said detecting comprises determining an amount of NMD-1 variant nucleic acid in said sample.

10. The method of claim 1, wherein said detecting NMD-1 variant nucleic acid differentiates NMD-1 variant from one or more of wild-type metallo-β-lactamase, VIM-1 metallo-β-lactamase, VIM-2 metallo-β-lactamase, non-NMD-1 variant nucleic acid, and nucleic acid from other multi-antibiotic resistant bacteria.

11-15. (canceled)

16. A kit for detecting NMD-1 variant in a sample, comprising: reagents for detecting a region of NMD-1 variant nucleic acid comprising the NDM-1 insertion and/or NMD-1 SNP to generate amplified NMD-1 variant nucleic acid.

17. The kit of claim 16, wherein said reagents comprise reagents for amplifying NMD-1 variant nucleic acid.

18. The kit of claim 16, wherein reagents for detecting NMD-1 variant nucleic acid comprise reagents for performing LATE-PCR on NMD-1 variant nucleic acid.

19. The kit of claim 16, wherein said reagents for amplifying NMD-1 variant nucleic acid comprise amplification primers.

20. The kit of claim 19, wherein said amplification primers hybridize to NMD-1 variant nucleic acid.

21. The kit of claim 29, wherein said amplification primers comprise SEQ ID NOS: 3 and 4, or a sequences having at least 70% identity therewith.

22. The kit of claim 20, wherein said amplification primers comprise SEQ ID NOS: 5 and 6, or a sequences having at least 70% identity therewith.

23. The kit of claim 20, wherein said amplification primers hybridize to a region of NMD-1 variant nucleic acid that differs from wild-type metallo-β-lactamase nucleic acids.

24. The kit of claim 16, wherein said reagents are contained within a reaction cartridge.

25. The kit of claim 24, wherein said reaction cartridge is configured to interact with a portable sample preparation and PCR instrument.


SEQUENCE LISTING (7 sequences)

SEQ ID NO: 1 - DNA, 415 bp, Escherichia coli
ccgcgtgctggtggtcgataccgcctggaccgatgaccagaccgcccagatcctcaactggatcaagcaggagatcaacctgccggtcgcgctggcggtggtgactcacgcgcatcaggacaagatgggcggtatggacgcgctgcatgcggcggggattgcgacttatgccaatgcgttgtcgaaccagcttgccccgcaagaggggatggttgcggcgcaacacagcctgactttcgccgccaatggctgggtcgaaccagcaaccgcgcccaactttggcccgctcaaggtattttaccccggccccggccacaccagtgacaatatcaccgttgggatcgacggcaccgacatcgcttttggtggctgcctgatcaaggacagcaaggccaagtcgctcggcaatctcggt

SEQ ID NO: 2 - DNA, 12 bp, Artificial Sequence
  Synthetic
ttcgccgccaat

SEQ ID NO: 3 - DNA, 20 bp, Artificial Sequence
  Synthetic
acagcctgactttcgccgcc

SEQ ID NO: 4 - DNA, 20 bp, Artificial Sequence
  Synthetic
aagcgatgtcggtgccgtcg

SEQ ID NO: 5 - DNA, 20 bp, Artificial Sequence
  Synthetic
cgacggcaccgacatcgctt

SEQ ID NO: 6 - DNA, 20 bp, Artificial Sequence
  Synthetic
sgcrcgsgcsswsgcsgcrw

SEQ ID NO: 7 - protein, 4 aa, Artificial Sequence
  Synthetic
FAAN